(19) United States
(12) Patent Application Publication
(10) Pub. No.: US 2025/0057404 A1
(43) Pub. Date: Feb. 20, 2025

(54) MINIATURE MULTISPECTRAL FLUORESCENCE IMAGING AND CELL COLLECTION PROBE

(71) Applicant: ARIZONA BOARD OF REGENTS ON BEHALF OF THE UNIVERSITY OF ARIZONA, Tucson, AZ (US)

(72) Inventors: TRAVIS SAWYER, TUCSON, AZ (US); BRIDGET SLOMKA, TUCSON, AZ (US)

(21) Appl. No.: 18/728,770

(22) PCT Filed: Jan. 12, 2023
(86) PCT No.: PCT/US2023/010708

§ 371 Date: Jul. 12, 2024

Related U.S. Application Data
(60) Provisional application No. 63/266,791, filed on Jan. 14, 2022.

(51) Int. Cl. A61B 1/07; A61B 1/06; A61B 5/00; A61B 10/02

(52) U.S. Cl. CPC A61B 1/07; A61B 1/0646; A61B 5/0071; A61B 10/02

(57) ABSTRACT

An imaging probe includes a housing, an illumination fiber, a camera, a band-stop filter, and a sample collector. The housing has a distal tip. The illumination fiber spans between the distal tip and a proximal end of the housing, and terminates at the distal tip, such that light carried by the illumination fiber illuminates a target in front of the distal tip. The camera captures an image in a field of view that overlaps a field of illumination of the illumination fiber. The band-stop filter is between the distal tip and the camera. The sample collector collects a sample from the target. And each of the illumination fiber, the camera, the band-stop filter, and the sample collector is at least partially inside the housing.

### RELATED APPLICATION

This application claims priority to U.S. Provisional Patent Application No. 63/266,791, filed Jan. 14, 2022, the disclosure of which is incorporated herein by reference in its entirety.

### BACKGROUND

Animal models, such as mouse models, are essential tools for studying cancer development and developing diagnostic and therapeutic techniques. One type of mouse model, the xenograft, involves implanting cultured tumor cells into the model and assessing the tumor growth and formation at later times. An alternate form of mouse model, the transgenic model, involves genetically manipulating the animal to produce naturally growing tumors, which commonly require much longer to grow than xenograft models. For tumors growing inside of the body, measurements often require sacrificing the animal and explanting the organ, which is difficult to perform and also prevents longitudinal monitoring of individual tumors.

One method of optical tumor assessment and diagnosis is to tag biomolecules of interest with a fluorescent contrast agent to enable detection with minimally invasive fluorescence endoscopy. The fluorescent marker can be introduced genetically, or through the application of dyes to bind to specific markers. However, utilizing fluorescence in vivo in a mouse model poses challenges due to the small and tortuous nature of the murine gastrointestinal tract.

### SUMMARY

Embodiments disclosed herein describe a device for imaging tissue at specific fluorescent wavelengths and collecting cells from areas of interest. In an embodiment, the device is an endoscope that includes a fiber optic lighting, a miniature imaging module, and a biopsy apparatus. The proximal end has a lighting apparatus that includes a broadband light source, a wavelength-adjustable filter wheel, a light guide, a collimator, and a fiber splitter. This endoscope may be configured with two parallel optical fibers that terminate at the distal tip of the endoscope. The distal end includes a miniature camera module and a multiband filter that blocks predetermined wavelength ranges tuned to biological fluorescent markers, such that signal from excitation wavelengths of light is not detected by the camera module. The multiband filter allows for the detection of a variety of clinically relevant fluorescence wavelengths without the need to remove the endoscope to change distal filters or devices, an advantage over similar devices. Images recorded from the detector are accessible to an operator via a cable connection to a computer.

In a first aspect, an imaging probe includes a device housing, illumination fiber optic cables, a camera, a wavelength filter, and a sample collector. The housing is sized for endoscopic and laparoscopic insertion and has a proximal end and a distal tip. The illumination fiber spans between the distal tip and a proximal end of the housing, and terminates at the distal tip, such that light carried by the illumination fiber illuminates a target in front of the distal tip. The camera captures an image in a field of view that overlaps a field of illumination of the illumination fiber. The band-stop filter is between the distal tip and the camera. The sample collector collects a sample from the target. And each of the illumination fiber, the camera, the wavelength filter, and the sample collector is at least partially inside the housing.

In a second aspect, an endoscope system includes the imaging probe described above, an illuminator, and an optical fiber. The imaging probe is disposed at a distal end of the endoscope system. The illuminator, disposed at a proximal end of the endoscope system, is operable to provide illumination having at least one selectable wavelength. The optical fiber has a first end optically coupled to the illuminator and a second end optically coupled to the illumination fiber.

### BRIEF DESCRIPTION OF THE FIGURES

FIG. 1 is a block diagram illustrating parts at a distal end and a proximal end of an endoscope, in an embodiment.

FIG. 2A and 2B illustrate cross-sectional views of an imaging probe of an endoscope system, in an embodiment.

FIG. 3 illustrates the lighting apparatus for an endoscope at a proximal end, in an embodiment.

FIG. 4A is a block diagram of a prototype endoscope system built for the experimental demonstration described herein.

FIG. 4B shows an in vivo application of an imaging probe shown in FIG. 4A.

FIG. 5 illustrates endoscope system of FIG. 4A with details on illumination configuration.

FIG. 6 shows details of imaging probe of FIG. 4B.

FIG. 7 illustrates an apparatus used for measuring the modulation transfer function of the endoscope system of FIG. 4A.

FIG. 8A shows an image of a standard slide used for the setup of FIG. 7, showing a blade edge that is back illuminated for measuring the modulation transfer function.

FIGS. 8B and 8C are plots showing the modulation transfer function measured using the setup of FIG. 7.

FIG. 9 shows images demonstrating effectiveness of the multiband interference filter in the imaging probe of FIG. 4B.

### DETAILED DESCRIPTION OF THE EMBODIMENTS

Fluorescence is the process by which a substance emits light when the substance has absorbed electromagnetic radiation. The emitted light, in most cases, has a longer wavelength than the absorbed radiation. The specific wavelengths of absorption and emission depend on the molecular and energetic structure of the material. Fluorescence is widely used in biomedical sciences to label specific cells or tissues, either through genetic manipulation or by dye application, giving a signature by which to track and identify the target of interest [1, 2]. Fluorescence labeling is highly specific, and targeted fluorescence markers have exhibited utility enabling cancer researchers to label cell lines of interest and track tumor growth in animal models [3].

Although ex vivo fluorescence microscopy and imaging are frequently applied to gastrointestinal (GI) cancer studies in murine models, it can be challenging to visualize real-time fluorescence of in vivo tissues inside the GI tract of these models due to their small and tortuous GI anatomy [4]. Real-time, in vivo methods of fluorescence imaging would enable the longitudinal studies of fluorescently tagged mice, reducing the animal burden for studies of cancer by eliminating the need for sacrifice at each study timepoint. Furthermore, enabling animal survival would ensure that the same individual subjects may be re-evaluated during cancer progression at later times in longitudinal studies, resolving challenges with consistency between timepoints that are commonly incurred when unique individuals must be sacrificed for each timepoint measurement. Additionally, to yield richer information, models can be tagged with multiple distinct fluorophores, each targeting unique markers. This utility is confirmed in the case of so-called Rainbow mice, previously utilized to visualize the spread of cancer oncogenes in intestinal epithelium by employing multiple fluorescent tags in each mouse [5].

The challenge of navigating small, tortuous lumens is not unique to GI cancer studies. For example, to traverse narrow lumens of other organs, such as human fallopian tubes, previous works have utilized sub-millimeter diameter fiber endoscopes with miniaturized biopsy systems [6, 7]. Despite their utility in the female reproductive system, the complex, expensive, and fragile components of these laser-illuminated, fiber-based imaging systems make them challenging to employ as GI cancer research tools for murine models. Similarly, the biopsy capabilities of these devices are unable to preserve tissue architecture, limiting utility of collected of cells to karyometry, rather than histology. In other works, multispectral scanning fiber endoscopes have been designed to suit the anatomical scale of murine colons [8]; however, these systems are limited to imaging as they are not equipped to biopsy suspected cancerous tissue for histological staining.

One aspect of the present embodiments is the realization that chip-on-tip endoscopes can acquire imaging data without the use of fragile fiber optics for signal transmission. The absence of said fiber optics improves system durability. Such a tool would be valuable in the context of animal research studies utilizing fluorescence markers.

The present disclosure details a miniature, multispectral, chip-on-tip fluorescence imaging tool to enable in vivo fluorescence imaging and biopsy of murine cancer models tagged with up to four unique fluorescent markers. This device, designed to fit in the working channel of commercially available gastroscopes, capitalizes on the miniaturization of both image sensors and multiband interference filters. This pairing results in a system capable of front-end filtering of incident light to enable imaging of multiple distinct bands (e.g., four bands) of fluorescence emission, without the need for filter tuning or other detector-end spectral selection methods. In concert, this system is designed to enable biopsy such that tissue architecture is collected for histological analysis at each timepoint of measurement over the course of longitudinal studies. We present the design of the system and characterize its performance through technical laboratory validation and demonstration on a fluorescent mouse model. The results indicate that the system performs with sufficient resolution and sensitivity to detect in vivo tissue fluorescence, and we demonstrate the ability to detect ex vivo tissue fluorescence.

Devices disclosed herein may be used in research setting to enable longitudinal studies of fluorescently labelled cancer cells in animal models. However, this device may be applied to other settings in which fluorescence imaging of sample or small spaces is required, especially in applications with more than one fluorescent emission wavelength. Accordingly, the term “target sample” is used herein and refers to a tangible physical medium capable of being rendered as an image.

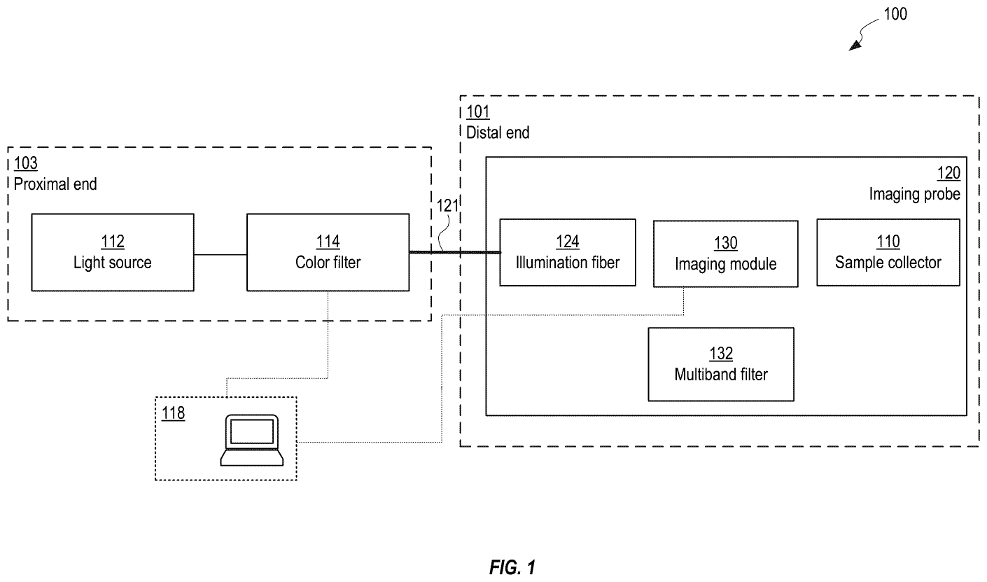

FIG. 1 illustrates an endoscope system 100. Endoscope system 100 includes a proximal end 103 and a distal end 101. Endoscope system 100 may also include a client device 118, which may be a display device for displaying data or an active device, such as a computer, that can execute a set of commands for endoscope system 100. Proximal end 103 provides illumination that may be at a specific wavelength, a plurality of specific wavelengths, or broadband. Additionally, proximal end 103 may be implemented by a single illumination element or a plurality of elements to provide the illumination. For example, in certain embodiments, proximal end 103 includes a light source 112 and a color filter 114. Light source 112 may be a broadband light source. For example, light source 112 may be, or include, a broadband light emitting diode (LED) or a plurality of narrow-band LEDs. Color filter 114 may be a bandpass filter that includes at least one spectral filter. Example center wavelengths of the spectral filter include 405, 488, 561, and 640 nanometers (nm). For example, when a fluorescent dye, such as coumarin, with an excitation wavelength of 402 nm is used for enhanced imaging of the target sample, a 405-nm spectral filter may be selected for color filter 114. Accordingly, color filter 114 may be communicably coupled to client device 118 for selecting a suitable spectral filter for the desired excitation wavelength.

In an alternate embodiment, proximal end 103 includes a plurality of narrow-band light sources. The plurality of narrow-band light sources may replace a broadband light source, such as light source 112, and may optically couple to a color filter. In an embodiment, the plurality of narrow-band light sources includes at least one narrow-band light source, such as a narrow-band LED. Example center wavelengths of the narrow-band LED include 405, 488, 561, and 640 nanometers. The narrow-band light sources may be communicably coupled to client device 118 for selecting a suitable narrow-band light source for the desired excitation wavelength.

Endoscope system 100 also includes an optical fiber 121. Filtered light from color filter 114 propagates in optical fiber 121 from proximal end 103 to distal end 101 for illuminating the target sample.

Distal end 101 includes an imaging probe 120. Imaging probe 120 includes an illumination fiber 124, an imaging module 130, and a sample collector 110. Illumination fiber 124 may be an extension of optical fiber 121 or may be optically coupled to optical fiber 121 with a coupler that may include at least one of an optical fiber coupler and a collimator. Sample collector 110 collects a sample while preserving tissue architecture. Sample collector 110 may be a cytology brush, a twisted wire, a miniature needle, a micro-brush, a sponge, a biopsy needle, a biopsy forceps, a microelectromechanical system, a liquid collector, or a combination thereof. In embodiments, when the target sample is comprised of cells, sample collector 110 may be capable of collecting at least 50,000 cells.

In certain embodiments, imaging module 130 is optically coupled to a multiband filter 132, which filters one or more ranges of wavelengths, where such filtering prevents certain spectral bands of light from reaching imaging module 130. In some embodiments, multiband filter 132 suppresses at least one wavelength range (e.g., center wavelength±10 nm) selected from wavelength ranges having center wavelengths of 405, 488, 561, and 640 nanometers. For example, when a fluorescent dye of coumarin is used, multiband filter 132 filters excitation wavelength of 405 nanometers while transmitting emission peak wavelength of 443 nanometers for detection by imaging module 130. Imaging module 130 may be communicably coupled to client device 118 and transmit imaging data for processing.

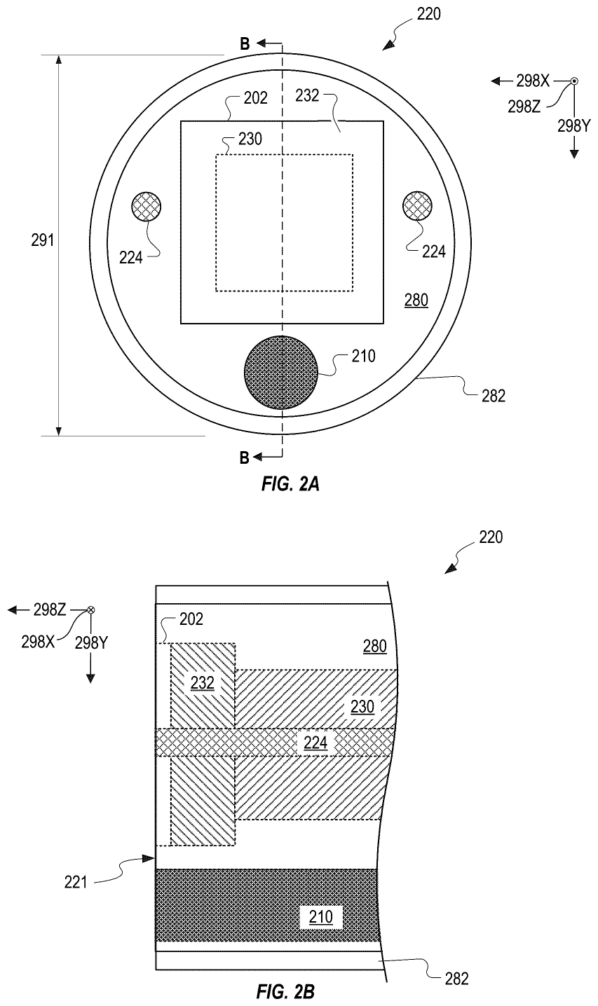

FIG. 2A and 2B illustrate cross-sectional views of an imaging probe 220 of an endoscope system. Imaging probe 220 is an example of imaging probe 120. FIGS. 2A and 2B are best viewed together in the following description. The view illustrated in FIG. 2A is parallel to a plane, hereinafter the x-y plane, formed by orthogonal axes 298X and 298Y, which are each orthogonal to an axis 298Z. Unless otherwise specified, depths of objects herein refer to the object's extent along axis 298Z. Herein, a reference to an axis x, y, or z refers to axes 298X, 298Y, and 298Z respectively. Also, herein, a width refers to an object's extent along the x axis, a height refers to an object's extent along the y axis, a thickness (or thinness) refers to an object's extent along the z axis, and vertical refers to a direction along the z axis. Also, herein, “above” refers to a relative position a distance away along the axis 298Z in the positive direction and “below” refers to a relative position a distance away along the axis 298Z in the negative direction. The section line B-B in FIG. 2A indicates the location of the orthogonal cross-sectional side view illustrated in FIG. 2B, which is parallel to the y-z plane.

In some embodiments, imaging probe 220 includes a housing 280, which may be a cylindrical shape, as shown in FIGS. 2A and 2B. Housing 280 may be rigid or flexible. In an embodiment, housing 280 is enclosed in a shell 282 that may be made of stainless-steel. Shell 282 may also be a flexible sheath. Imaging probe 220 has an outer diameter 291. In an embodiment, diameter 291 is less than three millimeters for laparoscopic or endoscopic insertion. As such, imaging probe 220 may be rigid or flexible depending on its application. Imaging probe 220 includes an illumination fiber 224, a multiband filter 232, an imaging module 230, and a sample collector 210, which are respective examples of illumination fiber 124, multiband filter 132, imaging module 130, and sample collector 110. Imaging module 230 may be a camera that includes an image sensor, an example of which includes a complementary metal-oxide semiconductor (CMOS) image sensor. Imaging module 230 is operable to capture an image or video of a sample in a field of view that overlaps with a field of illumination of illumination fiber 224. Sample collector 210 may include a movable part for collecting a sample. For example, sample collector 210 may extend and retract (in the z-direction) from the distal tip of imaging probe 220 to collect a sample. Sample collector 210 may also rotate to aid in collecting a sample. In embodiments, sample collector 210 is up to 0.65 millimeters in diameter. Imaging probe 220 may also include a cover plate 202, which may be made of glass.

Illumination fiber 224 is optically coupled to light source 112 or color filter 114 (FIG. 1) at proximal end for illuminating the target sample. In some embodiments, illumination fiber 224 includes more than one optical fiber at the distal tip for evenly illuminating the target sample. For example, FIG. 2A shows two illumination fibers 224 that may have split from an optical fiber, such as optical fiber 121, at the proximal end.

Imaging module 230 may be a chip-on-tip module capable of fluorescence imaging. For example, imaging module 230 may include a miniature CMOS image sensor. Imaging module 230 has dimensions that may be of up to 1 mm (width)×1 mm (height)×1.6 mm (depth). Imaging module 230 may have an imaging resolution of at least 249×250 pixels. In embodiments, imaging module 230 has a field of view of at least 90 degrees. Imaging module 230 may be capable of capturing images or video in the field of view and transferring the captured data to client device 118.

Multiband filter 232 is between a distal tip 221 of imaging probe 220 and imaging module 230. In some embodiments, multiband filter 232 is a band-stop filter that prevents a specific range of frequencies from passing to the output. For example, multiband filter 232 prevents excitation light (e.g., emitted by illumination fibers 224) from saturating the image data by not allowing the excitation light while allowing emission light to propagate to imaging module 230. The predetermined wavelength, or the stop band of the band-stop filter, may include, but are not limited to, any combination of center wavelengths of 405, 488, 561, and 640 nanometers, such that transmission of excitation wavelengths of commonly used fluorescent dyes into the imaging module 230 is suppressed.

Sample collector 210 is used to collect a target sample. Sample collector 210 may be movable to aid in collecting samples. For example, sample collector 210 may extend and/or rotate along z direction and may be controlled from the proximal end manually or by an instruction from a client device, such as client device 118.

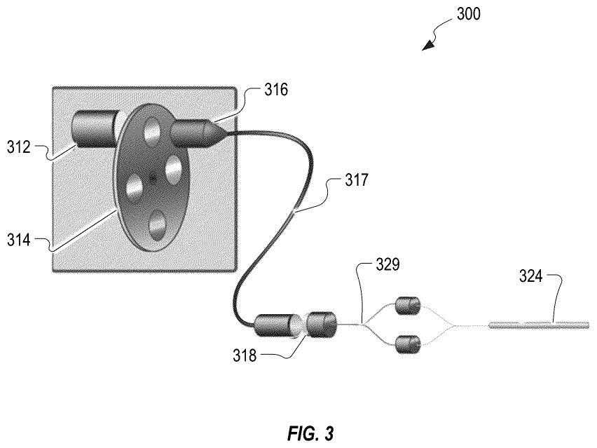

FIG. 3 illustrates a lighting apparatus 300. Lighting apparatus 300 may be disposed at the proximal end of endoscope system 100. Lighting apparatus 300 includes a light source 312 and a color filter 314, which are respective examples of light source 112 and color filter 114. Light source 312 may be a broadband light source or may comprise at least one narrow-band source. For example, light source 312 may generate a broad-spectrum light with wavelengths in the visible range, from 350 to 700 nanometers for example. Color filter 314 may be a filter wheel that includes at least one spectral filter at one of wavelengths of 405, 488, 561, and 640 nanometers. Color filter 314 may also pass a broad-spectrum light from light source 312 without color filtering. In embodiments, color filter 314 is communicably coupled to a client device (e.g., client device 118), such that the client device selects the illumination wavelength. For example, when fluorescent dye of coumarin is used. color filter 314 may be configured to pass light with center wavelength of 405-nanometer from light source 312 to generate the excitation light near 402-nanometer wavelength. In certain embodiments, one or both of light source 312 and color filter 314 is replaced with a wavelength-selectable light source.

Lighting apparatus 300 may also include an optical light guide 317, such as an optical fiber or a fiber bundle, for optically coupling the output of color filter 314 to an illumination fiber 324. Illumination fiber 324 may optically couple to illumination fiber 224 using optical couplers or splitters. Illumination fiber 324 may also extend to an imaging probe at distal end and become illumination fiber 224. Optical light guide 317 may include an optical receiver 316, such as a lens, for collecting the light from color filter 314. Lighting apparatus 300 may also include a fiber collimator 318 between optical light guide 317 and illumination fiber 324 for collimating the light. The collimated light may be split by a fiber splitter 329 into more than one illumination fibers 324.

### Experimental Demonstration

An endoscope system having a miniature fluorescence imaging probe (e.g., imaging probe 220), and equipped with a lighting apparatus (e.g., lighting apparatus 300) is described in a use scenario in an example below. The example describes a prototype endoscope system, an embodiment of endoscope system 100, that was fabricated as an imaging and biopsy device to image and sample fluorescently tagged, xenografted tumors as the tumors develop in mouse models. Specifically, described below are design and characterization of a device, which is an embodiment of imaging probe 220, and measurements of the modulation transfer function and ex vivo imaging performance. Such a device may be a valuable tool to advance cancer research in xenograft models, enabling the development of imaging biomarkers for cancer detection in a clinical setting without the need for exogenous contrast.

### 1. Device Fabrication

The fabrication requirements for the prototype endoscope system were determined in accordance with constraints introduced by the type of data to be acquired, such as the dimensional scale of the murine GI tract, and requirements for histological analysis of GI tissues, as summarized in Table 1 below.

TABLE 1

| Specification | Requirement | | Purpose |
|---|---|---|---|
| Max. Outer Diameter | 2.8 | mm | Minimize incision for laparoscopic |
| | | | introduction; ability to fit within |
| | | | working channel of commercial |
| | | | gastroscopes |
| Biopsy Type | Needle or Bite | | Preserve tissue architecture for |
| | | | histological analysis |
| Modalities included | Reflectance, | | Facilitate navigation and fluorophore |
| | fluorescence | | detection |
| Field of View | ≥50 | degrees | Ensure complete visibility of |
| | | | surrounding lumen walls |
| Pixel Resolution | >250 × 250 | px | Enable visualization of tissue |
| | | | morphology for lesion detection |
| Excitation wavelength | 405 ± 10 | nm | Excitation wavelength of Coumarin |
| ranges | 488 ± 10 | nm | Excitation wavelength of Cy2, GFP, |
| | | | AF488 |
| | 561 ± 10 | nm | Excitation wavelength of tdTomato, |
| | | | DsRed2 |
| | 640 ± 10 | nm | Excitation wavelength of Cy5, AF633 |
| Detection wavelength | 432-456 | nm | Detection of fluorescence emission for |
| ranges | | | Coumarin |
| | 519-529 | nm | Detection of fluorescence emission for |
| | | | Cy2, GFP, AF488 |
| | 595-605 | nm | Detection of fluorescence emission for |
| | | | tdTomato, DsRed2-C1 |
| | 680-735 | nm | Detection of fluorescence emission for |
| | | | Cy5, AF633 |
| Sterilization | Alcohol or soap with | | Ensure device sanitation appropriate |
| | water bath | | for surgical procedures |
| Working Distance | 5-15 | mm | Enable visualization along lumen and |
| | | | enable detection of masses |

| Imaging Mode | Video, photo | Enable real-time detection and data |
|---|---|---|
| | | recording |
| Illumination Type | narrowband via fiber | Reduction of cost and ease of |
| | optic | disposability |

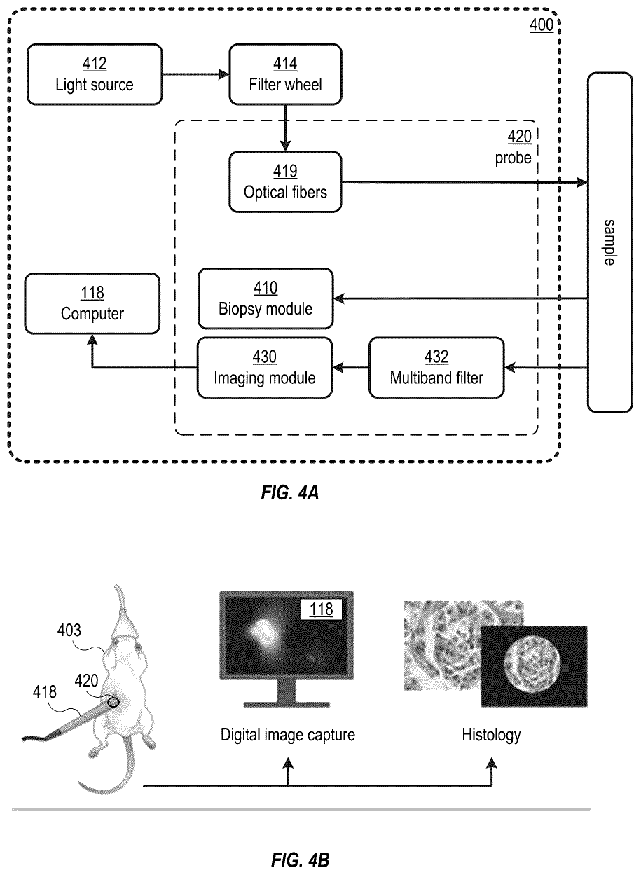

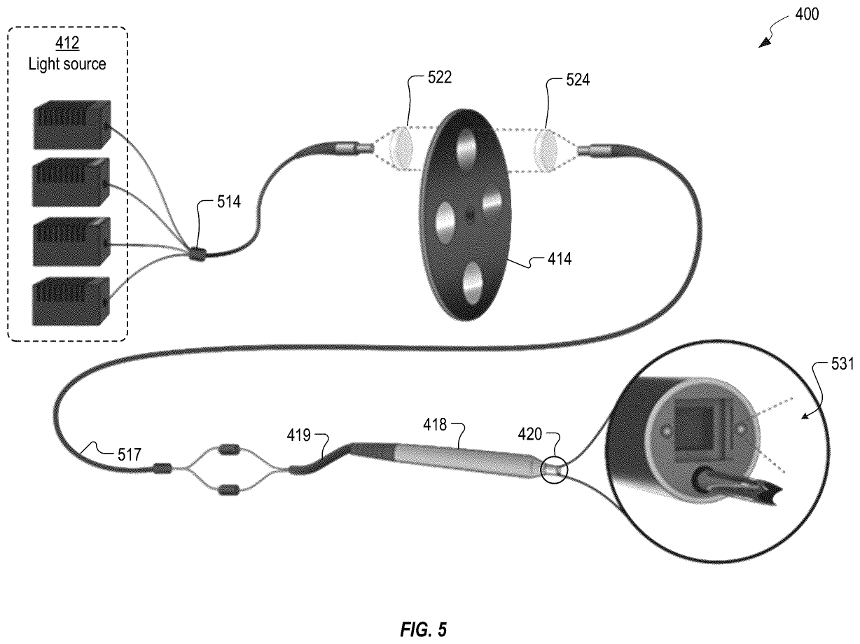

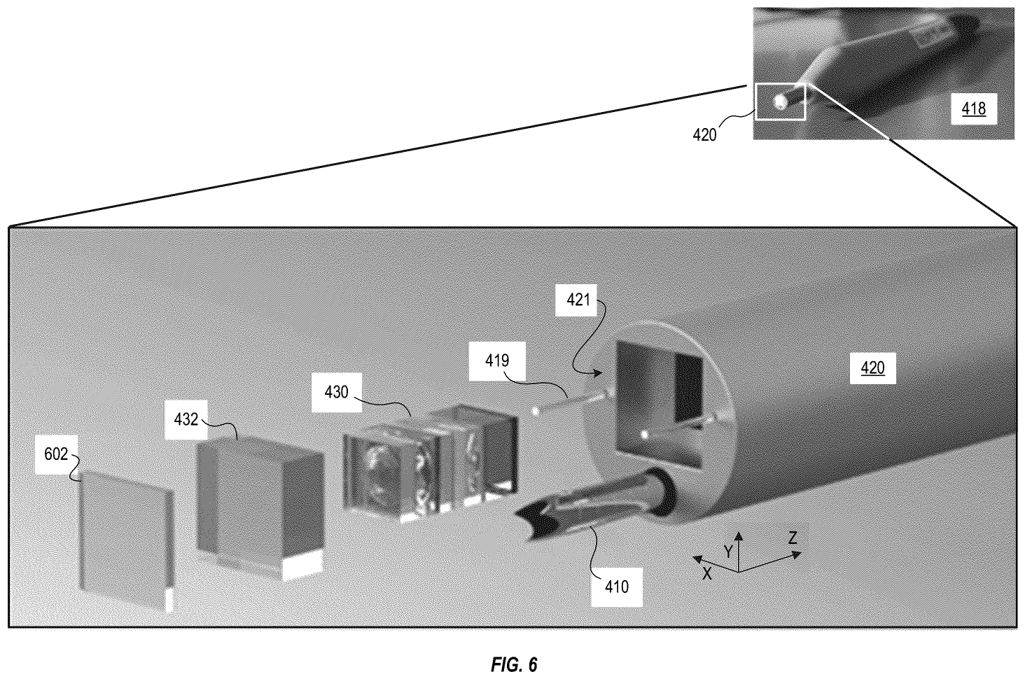

FIG. 4A is a block diagram of a prototype endoscope system 400 built for this experimental demonstration. Endoscope system 400 is an implementation of endoscope system 100 of FIG. 1 and includes all components described for endoscope system 100. FIG. 4B shows an in vivo application of an imaging probe 420 imaging a mouse 403. Imaging probe 420 is enclosed in a probe handle 418. Imaging probe 420 is an example of imaging probe 220, FIGS. 2A and 2B. FIG. 5 illustrates prototype endoscope system 400 with details on illumination configuration. FIG. 6 shows details of imaging probe 420 of FIGS. 4, 5A, and 5B. FIGS. 4A, 4B, 5 and 6 are best viewed together in the following description.

Prototype endoscope system 400 includes, at its proximal end, a light source 412, a 1-to-4 fanout fiber bundle 514, an aspheric condensing lens 522, a filter wheel 414, an additional aspheric condensing lens 524, a 1×2 multimode fiber coupler 517, and two multimode fibers 419. Light source 412 and filter wheel 414 are respective examples of light source 112 and color filter 114. At distal end, endoscope system 400 includes a probe handle 418 and imaging probe 420. Two multimode fibers 419 extend from the proximal end to the distal end and exit at the distal end of imaging probe 420 to produce illumination 531.

For the lighting apparatus, to provide sufficient illumination at each target wavelength, light source 412 includes four LED light sources, described in Table 2 below. The LED light sources are connected via a 400 μm core 1-to-4 fanout fiber bundle 514 to aspheric condensing lens 522. The output light is filtered through corresponding bandpass filters (Table 2) mounted in filter wheel 414 and focused with additional aspheric condensing lens 524 onto a 1×2 multimode fiber coupler 517, which pairs with two multimode fibers 419. Multimode fibers 419 extend to the distal end of the imaging probe and exit at distal tip 421.

TABLE 2

| Excitation | LED Light Source Nominal | Excitation Filter |
|---|---|---|
| Wavelength (nm) | Wavelength (nm) | Wavelength (nm) |
| 405 | 405 | 405 ± 10 |
| 488 | 490 | 488 ± 10 |
| 561 | 554 | 560 ± 10 |
| 640 | 625 | 640 ± 10 |

Imaging probe 420 includes a glass coverslip 602 (FIG. 6), a multiband interference filter 432, a miniature CMOS camera module 430, and multimode fibers 419. Imaging probe 420 further includes a biopsy needle 410, which is an example of sample collector 210. Imaging probe 420 is fabricated using a cylindrical housing of 2.7 mm in diameter by 10 mm in length with four through-holes. The terminal ends of two multimode fibers 419 are mounted in the lateral through-holes of the cylinder, and a miniature CMOS camera module 430, measuring 1.1 mm×1.1 mm (x×y), is positioned in the central through-hole of the printed cylinder such that the multiband interference filter 432 is placed directly in contact with the front of the camera lens of the camera module 430, facing outward from distal tip 421. For biopsy needle 410, a 25-gauge biopsy needle is mounted in the fourth through-hole, located below the camera module.

Multiband interference filter 432 is selected to suppress four distinct fluorescence excitation wavelength bands centered at 405 nm, 488 nm, 561 nm, and 640 nm, which correspond to fluorescent compositions Alexa Fluor 405 (AF405), Green Fluorescent Protein (GFP), Tandem dimer Tomato (tdTomato), and Cyanine 5 (Cy5), respectively. Multiband interference filter 432, however, allows transmission of fluorescent emission wavelengths centered at 420 nm, 510 nm, 580 nm, and 666 nm, corresponding to AF405, GFP, tdTomato, and Cy5, respectively. Multiband interference filter 432 is custom fabricated with a size of 1.5 mm×1.5 mm (x×y) to allow 90% transmission of fluorescence emission wavelengths while simultaneously preventing the transmission of fluorescence excitation wavelengths with an optical density of at least five.

The components of imaging probe 420 are then housed in a stainless-steel shell measuring 3 mm×14 mm (y×z). This housing is inserted into probe handle 418, a stylus-like stereolithography (SLA) 3D-printed shell using rigid resin. The housing is terminated with a flexible, SLA 3D-printed strain relief to protect the exiting illumination fibers and cables. All components are secured with a polyvinyl alcohol adhesive. The lighting system is controlled with a computer-interfaced driver and software. The camera module is computer-operated with an image processing system using an associated software.

### 2. Performance Validation

The system resolution of the prototype endoscope system 400 is assessed by measuring its Modulation Transfer Function (MTF). To derive the MTF of the system, data are collected in a configuration that simulated conditions during normal use, and the slanted-edge MTF analytical technique is employed.

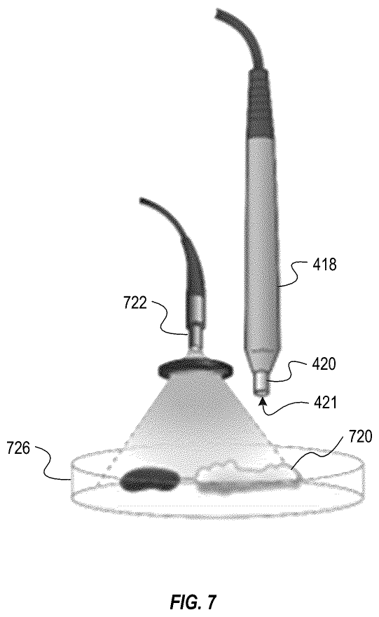

FIG. 7 illustrates an apparatus used for measuring the modulation transfer function of the endoscope system 400 of FIG. 4A. For preliminary validation of ex vivo fluorescence performance, the imaging system is configured with vertically positioned imaging probe 420 with the distal tip 421 2-3 cm above samples of ex vivo murine stomach tissue 720 modified to express ZSgreen in gastrin-secreting G cells. An external light source 722 is used to illuminate sections of stomach tissue 720 in a petri dish 726. Images are captured with both broadband unfiltered light for organ context and 488 nm filtered lighting for excitation of ZSgreen. The captured mages are then saved, and fluorescence detection data are enhanced and overlayed upon the white light images to create a composite image highlighting signal-producing regions of the GI tract.

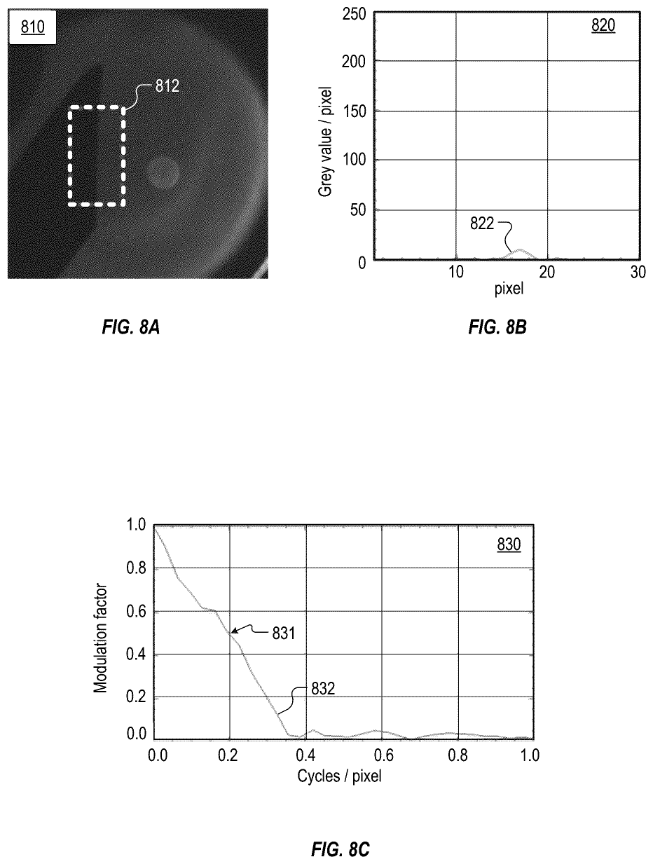

To perform the analysis, test images are captured of the interface of a slightly tilted, sharpened blade, and a fluorescently illuminated background slide that is back-illuminated with the 405 nm LED light source from Table 2. FIG. 8A shows an image 810 of a standard slide used for the setup of FIG. 7, showing a blade edge back illuminated for measuring the modulation transfer function. A highlighted rectangle in FIG. 8A indicates a region 812 selected for slanted-edge MTF analysis. Region 812 crossing the slanted edge interface of the sharpened blade and the fluorescently illuminated background slide is selected using the ImageJ plugin of Slanted Edge MTF [10] to subsequently yield the modulation transfer function of the system in cycles/pixel shown in FIG. 8C.

### 3. Results & Discussion

FIGS. 8B and 8C are plots 820 and 830 showing the modulation transfer function measured using the setup of FIG. 7. The endoscope system 400 with a miniature. multispectral, chip-on-tip imaging probe that enables fluorescence imaging of murine GI tracts, described above was used for this measurement. Designed as an ergonomic stylus to accommodate gloved hands, probe handle 418 of the probe is easy to handle and sufficiently encases all components including imaging probe 420. The directionality of the multiband interference filter 432 is determined pertinent to reduce penetrance by undesired wavelengths, and the angle of incidence impacted the effectiveness of the filter, with oblique angles allowing excess excitatory light passage.

Analysis of the slanted-edge MTF method yields the line spread function 822 shown in plot 820 in FIG. 8B and the MTF 832 shown in plot 830 in FIG. 8C. As shown in plot 830, an MTF₅₀ 831 of the system is 0.2 cycles/pixel. With a pixel size of 2.4 μm, this system is capable of resolving 83.3 cycles/mm in 50% of incidences, suggesting sufficient resolution for preliminary imaging of small, murine tumors. The cutoff frequency for this system was determined to be 0.35 cycles/pixel, or 145.8 cycles/mm. Resulting from the nature of the fluorescence emission of the backlit slide, the imaged interface in FIG. 8A does not exhibit a sharp, well-contrasted interface. Since the illumination is configured to simulate typical application of the imaging system, the contrast between the blade edge and fluorescing slide is less than that of the blade and a bright white background. The MTF values gained from this analysis report serve as conservative estimates of device functionality in dimly emissive environments.

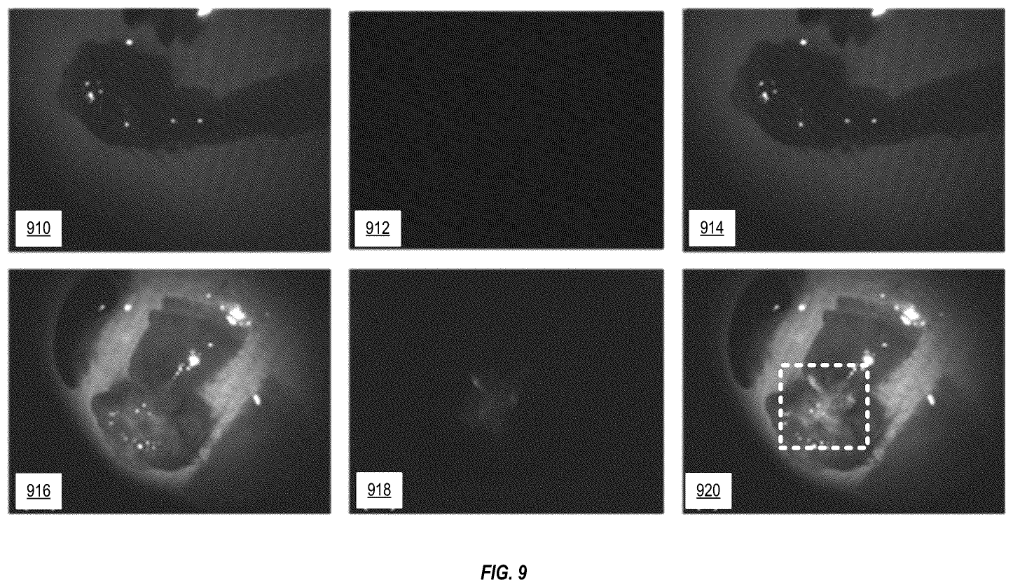

FIG. 9 shows images 910, 912, 914, 916, 918, and 920 demonstrating effectiveness of the multiband interference filter 432 in the imaging probe 420 using samples of ex vivo murine GI tissue. Multiband interference filter 432 effectively blocks detection of excitation wavelengths while passing emission photons from fluorescent marker ZSgreen. Image 910 is a broadband light image of wild type (WT) mouse kidney and stomach. Image 912 is an image of WT mouse kidney and stomach, taken at 488 nm, exhibiting no detectable fluorescent signal. Image 914 is a composite image of the WT broadband light and 488 nm images. Image 916 is a broadband light image of mouse kidney and ZSgreen-expressing stomach, with duodenum, enhanced for clarity. Image 918 is an image of mouse kidney and ZSgreen-expressing stomach, taken at 488 nm. Fluorescence is localized to the antrum. Image 920 is a composite image of the broadband light and 488 nm images for the ZSgreen-expressing mice, enhanced for clarity. A highlighted area in image 920 indicates the presence of fluorescence in the antrum of the stomach, the region in which gastrin-secreting G cells are located. Following the confirmation of fluorescent signal detection in ex vivo tissue, in vivo xenograft animal studies with fluorescently labeled cancer cell lines may be developed to further quantify the degree of fluorescence detection.

### Combinations of Features

Features described above as well as those claimed below may be combined in various ways without departing from the scope hereof. The following enumerated examples illustrate some possible, non-limiting combinations.

(A1) An imaging probe includes a housing, an illumination fiber, a camera, a band-stop filter, and a sample collector. The housing has a distal tip. The illumination fiber spans between the distal tip and a proximal end of the housing, and terminates at the distal tip, such that light carried by the illumination fiber illuminates a target in front of the distal tip. The camera captures an image in a field of view that overlaps a field of illumination of the illumination fiber. The band-stop filter is between the distal tip and the camera. The sample collector collects a sample from the target. And each of the illumination fiber, the camera, the band-stop filter, and the sample collector is at least partially inside the housing.

(A2) In embodiments of imaging probe (A1), the sample collector is operable to extend from the distal tip and rotate for collecting the sample.

(A3) In embodiments of either imaging probes (A1) or (A2), the sample collector is one of a cytology brush, a twisted wire, a miniature needle, a micro-brush, a sponge, a biopsy needle, a biopsy forceps, a microelectromechanical system, and a liquid collector.

(A4) In embodiments of any of imaging probes (A1)-(A3), the band-stop filter comprises a multiband interference filter.

(A5) In embodiments of any of imaging probes (A1)-(A4), the band-stop filter suppresses a fluorescence excitation wavelength of a fluorescent compound and allows a fluorescence emission wavelength of the fluorescent compound to pass through the band-stop filter.

(A6) In embodiments of imaging probe (A5), the fluorescent compound includes at least one of Alexa Fluor 405, Green Fluorescent Protein, tandem dimer Tomato, and Cyanine 5.

(A7) In embodiments of any of imaging probes (A1)-(A6), an outer diameter of the housing is less than three millimeters.

(A8) In embodiments of any of imaging probes (A1)-(A7), the housing is rigid or flexible.

(A9) Embodiments of any of imaging probes (A1)-(A8) further include a stylus that surrounds the imaging probe.

(B1) An endoscope system includes an illuminator, an optical fiber, and any one of imaging probes (A1)-(A8). The imaging probe is disposed at a distal end of the endoscope system. The illuminator, disposed at a proximal end of the endoscope system. The optical fiber has a first end optically coupled to the illuminator and a second end optically coupled to the illumination fiber.

(B2) Embodiments of system (B1) further include a display device communicatively coupled to the camera.

(B3) In embodiments of either system (B1) or (B2), the illuminator provides illumination having at least one spectral band.

(B4) In embodiments of system (B3), the at least one spectral band includes one of the following electromagnetic wavelengths: 405 nanometers, 488 nanometers, 561 nanometers, and 640 nanometers.

(B5) In embodiments of system (B4), the illumination includes an additional spectral band that includes electromagnetic wavelengths not overlapping the spectral band.

(B6) In embodiments of system (B5), the additional spectral band includes one of the following electromagnetic wavelengths: 405 nanometers, 488 nanometers, 561 nanometers, and 640 nanometers.

(B7) In embodiments of any of systems (B1)-(B6), the illuminator includes a broadband light source and a bandpass filter between the broadband light source and the optical fiber.

(B8) In embodiments of system (B7), the broadband light source includes a plurality of narrow-band light sources.

(B9) In embodiments of either system (B6) or (B7), the bandpass filter is a tunable bandpass filter.

(B10) In embodiments of any of systems (B6)-(B9), the bandpass filter has a transmission spectrum that includes at least one of 405 nanometers, 488 nanometers, 561nanometers, and 640 nanometers.

(B11) In embodiments of system (B10), a spectral width of the bandpass filter's passband does not exceed ten nanometers.

Changes may be made in the above methods and systems without departing from the scope of the present embodiments. It should thus be noted that the matter contained in the above description or shown in the accompanying drawings should be interpreted as illustrative and not in a limiting sense. Herein, and unless otherwise indicated the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

### REFERENCES

[1] Drummen, G. P. C., “Fluorescent probes and fluorescence (microscopy) techniques-illuminating biological and biomedical research,” in Molecules 17(12), Multidisciplinary Digital Publishing Institute (MDPI), pp. 14067-14090 (2012).
[2] Luo, S., Zhang, E., Su, Y., Cheng. T., and Shi, C., “A review of NIR dyes in cancer targeting and imaging,” in Biomaterials 32(29), Biomaterials, pp. 7127-7138 (2011).
[3] Sawyer, T. W., Koevary, J. W., Howard, C. C., Austin, O. J., Rice, P. F. S., Hutchens, G. V., Chambers, S. K., Connolly, D. C., and Barton, J. K., “Fluorescence and Multiphoton Imaging for Tissue Characterization of a Model of Post-Menopausal Ovarian Cancer,” Lasers in surgery and medicine 52(10), 993 (2020).
[4] Williams, J. M., Duckworth, C. A., Vowell, K., Burkitt, M. D., and Pritchard, D. M., “Intestinal Preparation Techniques for Histological Analysis in the Mouse,” Current protocols in mouse biology 6(2), 148-168 (2016).
[5] Boone, P. G., Rochelle, L. K., Ginzel, J. D., Lubkov, V., Roberts, W. L., Nicholls, P. J., Bock, C., Flowers, M. L., von Furstenberg, R. J., et al., “A cancer rainbow mouse for visualizing the functional genomics of oncogenic clonal expansion,” Nature Communications 10(1), (2019).

[6] Cordova, R., Kiekens, K., Burrell, S., Drake, W., Kmeid, Z., Rice, P., Rocha, A., Diaz, S., Yamada, S., et al., “Sub-millimeter endoscope demonstrates feasibility of in vivo reflectance imaging, fluorescence imaging, and cell collection in the fallopian tubes,” Journal of Biomedical Optics 26(07), (2021).
[7] Kiekens, K. C., Romano, G., Galvez, D., Cordova, R., Heusinkveld, J., Hatch, K., Drake, W., Kmeid, Z., and Barton, J. K., “Reengineering a falloposcope imaging system for clinical use,” Translational Biophotonics 2(4), (2020).
[8] Miller, S. J., Lee, C. M., Joshi, B. P., Gaustad, A., Seibel, E. J., and Wang, T. D.-S., “Targeted detection of murine colonic dysplasia in vivo with flexible multispectral scanning fiber endoscopy,” Journal of Biomedical Optics 17(2), 021103 (2012).
[9] Burns, P. D., “Slanted-Edge MTF for Digital Camera and Scanner Analysis,” in Soc. Imaging Sci. Technol. Image Process. Image Qual. Image Capture, Syst. Conf., 135-138 (2000).
[10] Mitja, C., Escofet, J., Tacho, A., and Revuelta, R., “Slanted Edge MTF-Image J,” 2011, <https://imagej.nih.gov/ij/plugins/se-mtf/index.html> (7 Jan. 2022).

What is claimed is:

1. An imaging probe, comprising:
a housing having a distal tip;
an illumination fiber that (i) spans between the distal tip and a proximal end of the housing, and (ii) terminates at the distal tip, such that light carried by the illumination fiber illuminates a target in front of the distal tip;
a camera that captures an image in a field of view that overlaps a field of illumination of the illumination fiber;
a band-stop filter between the distal tip and the camera; and
a sample collector that collects a sample from the target;
each of the illumination fiber, the camera, the band-stop filter, and the sample collector at least partially inside the housing.

2. The imaging probe of claim 1, the sample collector operable to extend from the distal tip and rotate for collecting the sample.

3. The imaging probe of claim 1, the sample collector being one of a cytology brush, a twisted wire, a miniature needle, a micro-brush, a sponge, a biopsy needle, a biopsy forceps, a microelectromechanical system, and a liquid collector.

4. The imaging probe of claim 1, the band-stop filter comprising a multiband interference filter.

5. The imaging probe of claim 1, the band-stop filter that suppresses a fluorescence excitation wavelength of a fluorescent compound and allows a fluorescence emission wavelength of the fluorescent compound to pass through the band-stop filter.

6. The imaging probe of claim 5, the fluorescent compound including at least one of Alexa Fluor 405, Green Fluorescent Protein, tandem dimer Tomato, and Cyanine 5.

7. The imaging probe of claim 1, an outer diameter of the housing being less than three millimeters.

8. The imaging probe of claim 1, the housing being rigid or flexible.

9. The imaging probe of claim 1, further comprising a stylus that surrounds the imaging probe.

10. An endoscope system, comprising:
the imaging probe of claim 1 disposed at a distal end of the endoscope system;
an illuminator, disposed at a proximal end of the endoscope system; and
an optical fiber having a first end optically coupled to the illuminator and a second end optically coupled to the illumination fiber.

11. The endoscope system of claim 10, further comprising a display device communicatively coupled to the camera.

12. The endoscope system of claim 10, the illuminator operable to provide illumination having at least one spectral band.

13. The endoscope system of claim 12, the at least one spectral band including one of the following electromagnetic wavelengths: 405 nanometers, 488 nanometers, 561 nanometers, and 640 nanometers.

14. The endoscope system of claim 13, the illumination including an additional spectral band that includes electromagnetic wavelengths not overlapping the spectral band.

15. The endoscope system of claim 14, the additional spectral band including one of the following electromagnetic wavelengths: 405 nanometers, 488 nanometers, 561 nanometers, and 640 nanometers.

16. The endoscope system of claim 10, the illuminator comprising:
a broadband light source; and
a bandpass filter between the broadband light source and the optical fiber.

17. The endoscope system of claim 16, wherein the broadband light source comprises a plurality of narrow-band light sources.

18. The endoscope system of claim 16, the bandpass filter being a tunable bandpass filter.

19. The endoscope system of claim 16, wherein the bandpass filter has a transmission spectrum that includes at least one of 405 nanometers, 488 nanometers, 561 nanometers, and 640 nanometers.

20. The endoscope system of claim 19, a spectral width of a passband of the bandpass filter not exceeding ten nanometers.
